(19) United States
(12) Patent Application Publication
(10) Pub. No.: US 2021/0361821 A1
(43) Pub. Date: Nov. 25, 2021

(54) MEDICAL DEVICE HAVING A PHOTOSENSITIZER AND RELATED METHODS

(71) Applicant: Becton, Dickinson and Company, Franklin Lakes, NJ (US)

(72) Inventors: Christopher Quach, Salt Lake City, UT (US); Gidon Ofek, Millcreek, UT (US); James Freasier, Salt Lake City, UT (US)

(21) Appl. No.: 17/224,661

(22) Filed: Apr. 7, 2021

Related U.S. Application Data
(60) Provisional application No. 63/027,750, filed on May 20, 2020.

(51) Int. Cl. A61L 15/44; A61F 13/00; A61M 39/20; A61M 39/10; A61N 5/06

(52) U.S. Cl. CPC A61L 15/44; A61F 13/00063; A61M 39/20; A61M 39/10; A61L 2300/606; A61N 5/0624; A61F 2013/00272; A61M 2039/1077; A61N 5/062

(57) ABSTRACT

A medical device may include a body and a photosensitizer integrated with the body. The medical device may passively resist colonization of bacteria under ambient light. The medical device may also actively resist colonization of bacteria by releasing reactive oxidative species (ROS) in response to administration of a light dose in a range of 0.5 J/cm² to 320 J/cm², for a duration between 1 second and 1 hour. The body may be formed by a base resin. The photosensitizer may be compounded with the base resin. The photosensitizer may be imbibed into the base resin. The medical device may include a coating disposed on a surface of the body. The photosensitizer may be disposed within the coating. The medical device may include a catheter adapter and a catheter extending distally from the catheter adapter. The catheter may be co-extruded with the photosensitizer and another material.

### RELATED APPLICATIONS

This application claims the benefit of U.S. Provisional Patent Application No. 63/027,750, filed on May 20, 2020, entitled “MEDICAL DEVICE HAVING A PHOTOSENSITIZER AND RELATED METHODS,” which is incorporated herein in its entirety.

### BACKGROUND

There are different kinds of photosensitizers, each one with a specific excitation wavelength. During photosensitizer photoactivation, an excitable electron is promoted to a higher energy level, and the photosensitizer reaches a first excited singlet state. The first excited singlet state decays to a lower energy level by emitting fluorescence or, alternatively, by a process of intersystem crossing to a state named triplet. The triplet state is long lived and, because of this feature, it is able to react with triplet oxygen molecules and other biomolecules or to emit phosphorescence. When the photosensitizer in the triplet state transfers energy to triplet oxygen, very reactive species called singlet oxygen are produced. Both the photosensitizer in the triplet state and the singlet oxygen are unstable molecules and for this reason they are responsible, in biological tissues, for the damage to polyunsaturated lipids, nucleic acids, and protein after photosensitizer irradiation.

A direct reaction between the photosensitizer in the triplet state and a biomolecule is called a Type 1 reaction and this process leads to photosensitizer blanching. Production of the singlet oxygen by the photosensitizer in the triplet state is called a Type 2 reaction and regenerates the photosensitizer in the ground state. Although both the Type 1 reaction and the Type 2 reaction can damage cells, more significant damage is induced by the Type 2 reaction than the Type 1 reaction.

Catheters are traditionally used to infuse fluids, such as saline solution, various medicaments, and/or total parenteral nutrition into a patient. Such catheters may also be used to withdraw blood from a patient, and/or monitor various parameters of the patient's vascular system. To introduce a catheter into a patient, an introducer needle may be used, which may include a sharp distal tip. The catheter may include an over-the-needle peripheral intravenous (“IV”) catheter mounted over the introducer needle. An inner surface of the catheter may tightly engage an outer surface of the introducer needle to prevent catheter peel back and facilitate insertion of the catheter into a blood vessel. The sharp distal tip of the introducer needle may extend beyond the distal tip of the catheter to enable insertion of the catheter at a shallow angle through skin of the patient and into the blood vessel.

To verify proper placement of the needle and the catheter in the blood vessel, the clinician may confirm the presence of “flashback” blood in a flashback chamber associated with the catheter and needle assembly. Once proper placement is confirmed, the clinician may then apply pressure to the blood vessel to occlude the vessel, thereby reducing further blood flow through the introducer needle and catheter. The clinician may then withdraw the needle from the catheter to enable continued access to the blood vessel through the catheter.

The catheter is susceptible to bacteria, which may infect and harm the patient. Similarly, other vascular access devices, as well as ultrasound devices, dressings, pumps, and other medical devices that come into close proximity with the patient may be susceptible to bacteria, which may infect and harm the patient. There is a need for a material that is capable of increasing antimicrobial resistance of medical devices.

The subject matter claimed in the present disclosure is not limited to embodiments that solve any disadvantages or that operate only in environments such as those described above. Rather, this background is only provided to illustrate one example technology area where some implementations described in the present disclosure may be practiced.

### SUMMARY

The present disclosure relates generally to medical devices and related methods that facilitate decontamination and antimicrobial resistance. In some embodiments, a medical device may include a body and a photosensitizer integrated with the body. In some embodiments, the photosensitizer may include methylene blue, new methylene blue, Nile blue, rose bengal, toluidine blue O, crystal violet, or another suitable photosensitizer. In some embodiments, the body may include multiple photosensitizers integrated with the body.

The photosensitizer may be integrated with the body in various ways. For example, the body may be formed by a base resin, and the photosensitizer may be compounded with the base resin. In some embodiments, the base resin may include polyphenylsulfone, polyurethane, or silicone. In some embodiments, a concentration of the photosensitizer within the base resin may be between 0.05% and 5%.

As another example, the medical device may include a coating disposed on a surface of the body, and the photosensitizer may be disposed within the coating. In some embodiments, a concentration of the photosensitizer within the coating may be between 0.05% and 5%. As yet another example, the medical device may include a catheter adapter and a catheter extending distally from the catheter adapter. In these embodiments, the catheter may be co-extruded with the photosensitizer and another material. As a further example, the body may be formed by the base resin, and the photosensitizer is imbibed into the base resin.

In some embodiments, the medical device may include a needleless connector or a cap. In some embodiments, the medical device may include an ultrasound transducer. In some embodiments, the medical device may include a dressing, a surgical mesh, a pump, or another suitable medical device.

In some embodiments, a method of disinfection of the medical device may include providing the medical device. In some embodiments, the method may include administering to the photosensitizer integrated with the body a light dose in a range between 0.5 J/cm² and 320 J/cm². In some embodiments, the light dose may be administered for a duration between 1 second and 1 hour.

In some embodiments, the method may include integrating the photosensitizer with the body of the medical device. In some embodiments, integrating the photosensitizer with the body of the medical device may include compounding the photosensitizer with the base resin. In some embodiments, integrating the photosensitizer with the body of the medical device may include applying the coating on a surface of the medical device. In some embodiments, the medical device may include the catheter adapter and the catheter, and integrating the photosensitizer with the body of the medical device may include co-extruding the catheter with the photosensitizer and another material. In some embodiments, integrating the photosensitizer with the body of the medical device may include imbibing the photosensitizer into the base resin.

It is to be understood that both the foregoing general description and the following detailed description are examples and explanatory and are not restrictive of the present disclosure, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of some embodiments of the present disclosure. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example medical device, according to some embodiments;

FIG. 1B is a cross-sectional view of the medical device of claim 1A, illustrating an example coating, according to some embodiments;

FIG. 2 is an upper perspective view of another example medical device, according to some embodiments;

FIG. 3 is an upper perspective view of another example medical device, according to some embodiments;

FIG. 4 is an upper perspective view of another example medical device, according to some embodiments;

FIG. 5A is an upper perspective view of another example medical device, according to some embodiments;

FIG. 5B is an enlarged upper perspective view of the medical device of FIG. 2A, according to some embodiments; and

FIG. 6 is a schematic diagram of an example chamber to provide a light dose to a medical device, according to some embodiments.

### DETAILED DESCRIPTION

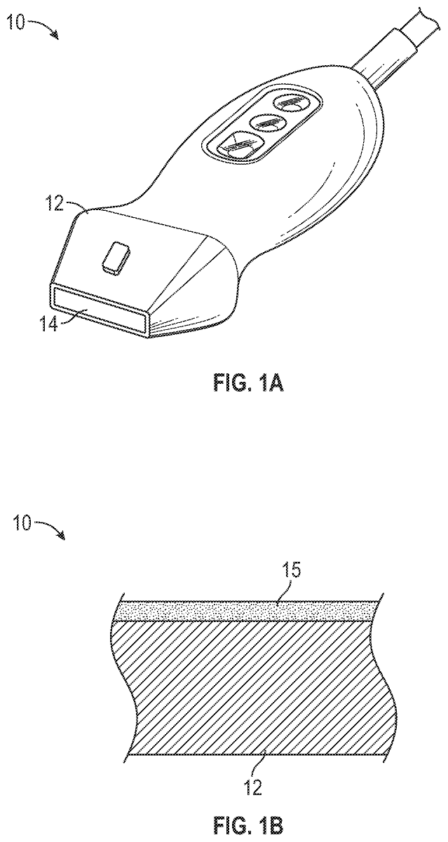

Referring now to FIG. 1A, a medical device 10 is illustrated. As illustrated, for example, in FIG. 1, the medical device 10 may include an ultrasound transducer. In other embodiments, the medical device 10 may include a catheter, a catheter adapter, a connector, a cap, an ultrasound transducer, a dressing, a surgical mesh, a pump, or another suitable medical device that comes into contact or close proximity with a patient.

In some embodiments, the medical device 10 may include a body 12 and a photosensitizer integrated with the body 12. In some embodiments, the body 12 may include any portion of the medical device 10 that contacts or is near the patient. In some embodiments, the medical device 10 may include the ultrasound transducer, and the body 12 may include a distal end 14 of the ultrasound transducer. In some embodiments, the photosensitizer may include methylene blue, new methylene blue, Nile blue, rose bengal, toluidine blue O, crystal violet, or another suitable photosensitizer.

In some embodiments, the photosensitizer may be integrated with the body 12 in various ways. In some embodiments, the body 12 may be formed by a base resin, and the photosensitizer may be compounded with the base resin. In some embodiments, the base resin may include polyphenylsulfone, polyurethane, silicone, or another suitable base resin. In some embodiments, a concentration of the photosensitizer within the base resin may be between 0.05% and 5%.

In some embodiments, the body 12 may be formed by the base resin, and the photosensitizer may be imbibed into the base resin. In some embodiments, imbibing may include dissolving the photosensitizer in methyl ethyl ketone (MEK), tetrahydrofuran (THF), or another suitable solvent to form a solution. In some embodiments, imbibing may include exposing the body 12 to the solution such that the body 12 absorbs the solution and swells. In some embodiments, the body 12 may be co-extruded with the photosensitizer and another material using a die.

In some embodiments, the body 12 may actively resist colonization of bacteria by releasing reactive oxidative species (ROS) in response to a light dose. In some embodiments, the light dose, which may be in a range between 0.5 J/cm² and 320 J/cm², may be administered to the photosensitizer integrated with the body 12. In some embodiments, the light dose may be administered for a duration between 1 second and 1 hour. In some embodiments, the light dose may be red light (about 700-635 nm), yellow light (about 590-560 nm) or ultraviolet (UV) light, such as, for example, UV C light. In some embodiments, the body 12 may passively resist colonization of bacteria under ambient light, which may be administered to the photosensitizer integrated with body 12. In some embodiments, the photosensitizer may produce lower levels of ROS in response to the ambient light than the photosensitizer produces in response to the light dose in the range between 0.5 J/cm² and 320 J/cm².

In some embodiments, the light dose may activate the photosensitizer, and the photosensitizer may reach a first excited singlet state as an excitable electron is promoted to a higher energy level. The first excited singlet state may decay to a lower energy level by a process of intersystem crossing to a state named triplet. The triplet state may be long lived and, because of this feature, the photosensitizer is able to react with triplet oxygen molecules and other biomolecules. When the photosensitizer in the triplet state transfers energy to triplet oxygen, an ROS called singlet oxygen may be produced. Both the photosensitizer in the triplet state and the singlet oxygen are unstable molecules and for this reason they may provide decontamination and antimicrobial resistance to the body 12. In some embodiments, varying wavelengths of light may be administered to the body 12 at different times and/or the body 12 may include multiple photosensitizers, which may be activated in response to different wavelengths of light.

Referring to FIG. 1B, in some embodiments, the medical device 10 may include a coating 15, which may be disposed on all or a portion of a surface of the body 12. In these embodiments, the photosensitizer may be disposed within the coating 15. In some embodiments, integrating the photosensitizer with the body 12 of the medical device 10 may include applying the coating 15 on the surface of the medical device 10. In some embodiments, a concentration of the photosensitizer within the coating 15 may be between 0.05% and 5%. In some embodiments, the photosensitizer may be dissolved in urethane, polyurethane, or another suitable solvent and may harden on the body 12 to form the coating 15.

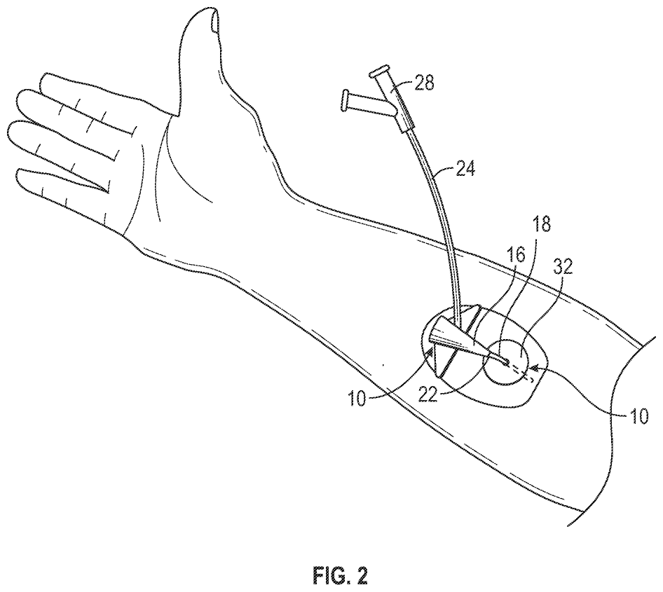

Referring now to FIG. 2, in some embodiments, the body 12 of the medical device 10 may include a catheter adapter 16 and/or a catheter 18 extending distally from the catheter adapter 16. In some embodiments, the photosensitizer may be integrated with the body 12 via one or more of the methods discussed with respect to FIG. 1. Thus, in some embodiments, the photosensitizer may be compounded with a base resin, disposed within a coating on the body 12, imbibed into the base resin, or co-extruded with another material. In some embodiments, the catheter 18 may be co-extruded with the photosensitizer and another material. In some embodiments, the catheter 18 may include a peripheral intravenous catheter, a midline catheter, or a peripherally-inserted central catheter. In some embodiments, the catheter 18 may include a proximal end 20 secured within the catheter adapter 16 and a distal end 22 configured to insert into vasculature of the patient.

In some embodiments, an extension tube 24 may extend from the catheter adapter 16. In some embodiments, a distal end of the extension tube 24 may be integrated with or coupled to a side port 26 of the catheter adapter 16. In some embodiments, a proximal end of the extension tube 24 may be integrated with or coupled to an adapter 28, which may include a Y-adapter or another suitable adapter.

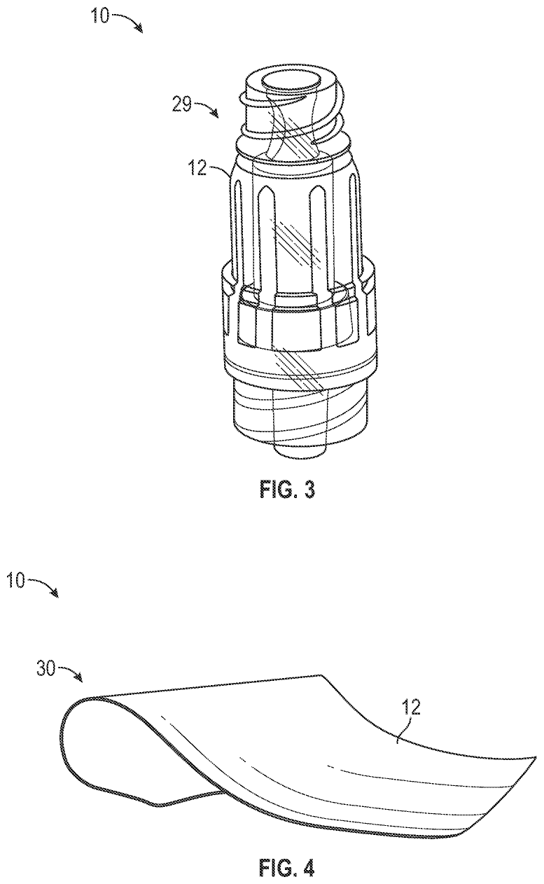

Referring now to FIG. 3, in some embodiments, the medical device 10 may include a connector 29, which may include the body 12. In some embodiments, the connector 29 may include a needleless connector, such as, for example, a SMARTSITE™ needle-free valve available from Becton, Dickinson & Company of Franklin Lakes, N.J. In some embodiments, the connector 29 may be coupled to the adapter 28 (see, for example, FIG. 2), another adapter coupled to a particular catheter adapter, a port of the particular catheter adapter, or another suitable location. In some embodiments, the photosensitizer may be integrated with the body 12 via one or more of the methods discussed with respect to FIG. 1. Thus, in some embodiments, the photosensitizer may be compounded with a base resin, disposed within a coating on the body 12, imbibed into the base resin, or co-extruded with another material.

Referring now to FIG. 4, in some embodiments, the medical device 10 may include a surgical mesh 30, which may include the body 12. In some embodiments, the photosensitizer may be integrated with the body 12 via one or more of the methods discussed with respect to FIG. 1. Thus, in some embodiments, the photosensitizer may be compounded with a base resin, disposed within a coating on the body 12, imbibed into the base resin, or co-extruded with another material.

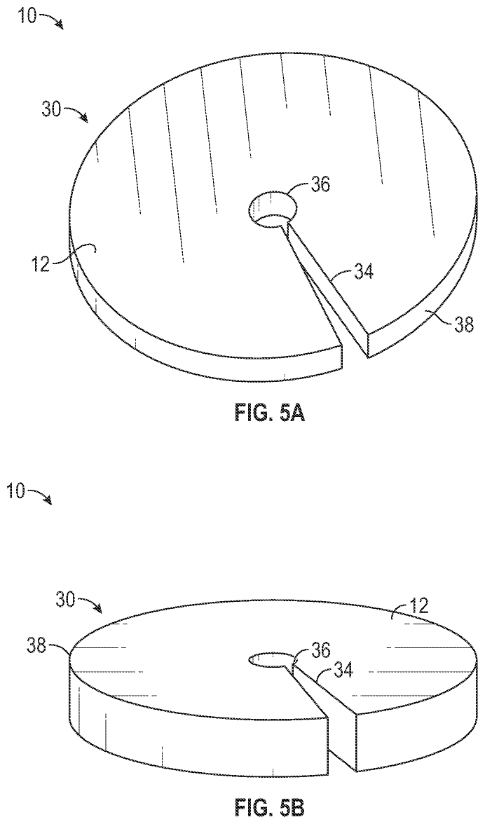

Referring now to FIGS. 5A-5B, in some embodiments, the medical device 10 may include a dressing 32 for use with a medical device inserted into the vasculature of the patient via a skin insertion or puncture site, which may include the body 12. In some embodiments, the photosensitizer may be integrated with the body 12 via one or more of the methods discussed with respect to FIG. 1.

In some embodiments, the dressing 32 may be configured for use with the medical device, such as the catheter 18 (see, for example, FIG. 2), that has punctured the skin of a patient and has a portion of the medical device protruding from the skin. In some embodiments, the body 12 may include a slit 34 configured to enable the body 12 to be placed around the medical device and on a surface of the skin such that the dressing body surrounds and contacts the skin insertion or puncture site. In some embodiments, the slit 34 may be formed in the body 12 by cutting, punching, or other similar mechanical forming technique. In some embodiments, a width of the slit 34 may be adapted to facilitate installation over the medical device already installed within the vasculature of the patient. In some embodiments, the slit 34 may enable the dressing 32 to fully surround the medical device at the skin insertion or puncture site.

In some embodiments, the body 12 may take any geometric shape. In some embodiments, the body 12 may be disk-shaped. In some embodiments, a shape of the body 12 may include an oval, triangle, square, rectangle, pentagon, hexagon, octagon, etc. In some embodiments, the body 12 may be fabricated of any physiologically compatible material that is capable of being impregnated or imbibed with the photosensitizer. In some embodiments, the body 12 may be constructed of oxidized cellulose foam, collagen fibrils, alginate hydrogel, or another suitable material.

In some embodiments, the body 12 may include an aperture 36 for reception of the medical device. In some embodiments, the slit 34 may extend from the aperture 36 to an outer perimeter 38 of the body 12. In some embodiments, the slit 34 may enable the body 12 to fully surround and contact the skin insertion site, through which the medical device, such as, for example, the catheter 18 (see FIG. 2), may pass.

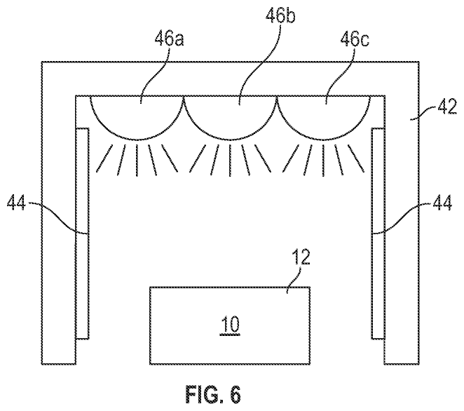

Referring now to FIG. 6, a chamber 40 to provide a light dose to the medical device 10 is illustrated, according to some embodiments. In some embodiments, the chamber 40 may be disposed within a cover 42, such as a box, container, etc. In some embodiments, one or more sides of the chamber 40 may include a mirror 44. In some embodiments, one or more light sources 46 may be disposed within the chamber 40. For example, the light sources may include one or more of: a first light source 46a, a second light source 46b, and a third light source 46c (which may be referred to in the present disclosure as “light sources 46”).

In some embodiments, each of the light sources 46 may emit a light dose having a particular wavelength of light. In some embodiments, the first light source 46a may emit a first wavelength, the second light source 46b may emit a second wavelength, and the third light source 46c may emit a third wavelength. For example, the first light source 46a may emit UVC light, the second light source 46b may emit red light, and the third light source 46c may emit yellow light. In some embodiments, the light sources 46 may include light emitting diodes (LEDs). In some embodiments, the light sources 46 may be activated at different times from each other and/or for different durations. In some embodiments, in response to activation of the light sources 46 different photosensitizers integrated with the body 12 may be activated, leading to free radical production and decontamination of the medical device 10 disposed within the chamber 40.

All examples and conditional language recited in the present disclosure are intended for pedagogical objects to aid the reader in understanding the present disclosure and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the present disclosure.

1. A medical device, comprising:
a body; and
a photosensitizer integrated with the body.

2. The medical device of claim 1, wherein the body is formed by a base resin, wherein the photosensitizer is compounded with the base resin.

3. The medical device of claim 2, wherein the base resin comprises polyphenylsulfone, polyurethane, or silicone.

4. The medical device of claim 2, wherein a concentration of the photosensitizer within the base resin is between 0.05% and 5%.

5. The medical device of claim 1, further comprising a coating disposed on a surface of the body, wherein the photosensitizer is disposed within the coating.

6. The medical device of claim 5, wherein a concentration of the photosensitizer within the coating is between 0.05% and 5%.

7. The medical device of claim 1, wherein the medical device comprises a catheter adapter and a catheter extending distally from the catheter adapter.

8. The medical device of claim 7, wherein the catheter is co-extruded with the photosensitizer and another material.

9. The medical device of claim 7, wherein the medical device comprises a needleless connector or a cap.

10. The medical device of claim 1, wherein the body is formed by a base resin, wherein the photosensitizer is imbibed into the base resin.

11. The vascular access device of claim 9, wherein the base resin comprises polyphenylsulfone, polyurethane, or silicone.

12. The medical device of claim 1, wherein the photosensitizer comprises methylene blue, new methylene blue, Nile blue, rose bengal, toluidine blue O, or crystal violet.

13. The medical device of claim 1, wherein the medical device comprises an ultrasound transducer.

14. The medical device of claim 1, wherein the medical device comprises a dressing.

15. A method of disinfection of a medical device, the method comprising:
providing a medical device, wherein the medical device comprises a body and a photosensitizer integrated with the body; and
administering to the photosensitizer integrated with the body of the medical device a light dose in a range of 0.5 J/cm² to 320 J/cm², for a duration between 1 second and 1 hour.

16. The method of claim 15, further comprising integrating the photosensitizer with the body of the medical device.

17. The method of claim 16, wherein the body is formed by a base resin, wherein integrating the photosensitizer with the body of the medical device comprises compounding the photosensitizer with a base resin.

18. The method of claim 16, wherein integrating the photosensitizer with the body of the medical device comprises applying a coating on a surface of the body, wherein the photosensitizer is disposed within the coating.

19. The method of claim 16, wherein the medical device comprises a catheter adapter and a catheter extending distally from the catheter adapter, wherein integrating the photosensitizer with the body of the medical device comprises co-extruding the catheter with the photosensitizer and another material.

20. The method of claim 16, wherein the medical device comprises a base resin, wherein integrating the photosensitizer with the body of the medical device comprises imbibing the photosensitizer into the base resin.
